(19) United States
(12) Patent Application Publication
(10) Pub. No.: US 2021/0378674 A1
(43) Pub. Date: Dec. 9, 2021

(54) Ligation Clips for Forming Purse Strings for Anastomoses Procedures and Method for Performing Anastomoses Procedures

(71) Applicant: Covidien LP, Mansfield, MA (US)

(72) Inventors: Justin Thomas, New Haven, CT (US); Jacob C. Baril, Norwalk, CT (US); Garrett P. Ebersole, Hamden, CT (US); Saumya Banerjee, Hamden, CT (US); Matthew A. Dinino, Newington, CT (US); Roy J. Pilletere, Middletown, CT (US)

(21) Appl. No.: 17/317,462

(22) Filed: May 11, 2021

Related U.S. Application Data
(60) Provisional application No. 63/034,073, filed on Jun. 3, 2020.

(51) Int. Cl. A61B 17/122; A61B 17/115; A61B 17/128

(52) U.S. Cl. CPC A61B 17/122; A61B 2017/1142; A61B 17/128; A61B 17/1155

(57) ABSTRACT

A ligation clip includes first and second beams that are movable in relation to each other from an open position to a clamped position to define an opening that is dimensioned to be received about end portions of vessel portions to be joined together during an anastomosis procedure to secure the end portions to an anvil shaft and/or an anvil retainer of a circular stapling device. The use of ligation clips to perform this function obviates the need to form a purse suture in the end portions of the vessel portions during the anastomosis procedure and reduces the amount of time required to perform an anastomosis procedure.

### CROSS-REFERENCE TO RELATED APPLICATION

This application claims the benefit of and priority to U.S. Provisional Patent Application No. 63/034,073, filed Jun. 3, 2020, which is incorporated by reference herein in its entirety.

### FIELD

This disclosure is directed ligation clips, and more particularly, to ligation clips for forming a purse string during an anastomosis procedure and to a method of performing an anastomosis using the ligation clips.

### BACKGROUND

Surgical anastomosis is an artificial connection of two vessel portions made by a surgeon and is required when a diseased portion of a vessel, e.g., artery, vein, or intestine, is removed from the vessel and remaining vessel portions must be rejoined. Typically, a circular stapling device is used to rejoin the vessel portions. In such a procedure, a purse string suture is formed in an end of each of the vessel portions and the purse string sutures are tightened to secure the ends of the vessel portions about an anvil shaft of an anvil of the circular stapling device and about an anvil retainer of the circular stapling device.

A purse string suture is a suture that is stitched into an end portion of the vessel portions and used as a drawstring to tighten the end portion about the anvil shaft and/or anvil retainer. Once the end portion of each of the vessel portions is tightly secured about the anvil shaft and/or the anvil retainer of the circular stapling device, the anvil of the circular stapling device is approximated with a staple cartridge of the circular stapling device to clamp the vessel portions to each other between the anvil and the staple cartridge. The circular stapling device is then fired to secure the vessel portions together with staples and to remove excess tissue from within a lumen defined by the joined vessel portions.

The process of forming a purse string suture in each end portion of the vessel portions is time consuming and increases the amount of time a patient must remain under anesthesia. This increases the trauma the patient must endure and increases the likelihood of infection.

A continuing need exists in the surgical arts for a method and device for quickly forming a purse string in the end portions of the vessel portions to be joined during an anastomosis procedure to reduce the time required to perform a surgical procedure.

### SUMMARY

One aspect of the disclosure is directed to a ligation clip including a first beam and a second beam. The first beam has first and second end portions and a first clamping surface. The second beam has first and second end portions and a second clamping surface. The first beam is movable in relation to the second beam to move the ligation clip from an open position to a clamped position. In the clamped position, the first and second clamping surfaces of the first and second beams define an opening that is dimensioned to receive an anvil shaft of a surgical stapling device to secure a vessel about the anvil shaft.

In aspects of the disclosure, each of the first end portions of the first and second beams includes a latch portion. The latch portions of the first and second beams engage each other when the ligation clip is in the clamped position to retain the ligation clip in the clamped position.

In some aspects of the disclosure, the diameter of the opening defined by the first and second clamping surfaces of the ligation clip when the ligation clip is in the clamped position is from about 0.3 inches to about 0.6 inches.

In certain aspects of the disclosure, the opening has a circular configuration.

In aspects of the disclosure, the opening has an oval configuration.

In some aspects of the disclosure, the opening has a rectangular configuration.

In certain aspects of the disclosure, each of the latch portions is hook shaped.

In aspects of the disclosure, the first and second beams are coupled together by a central hinge portion.

In some aspects of the disclosure, the central hinge portion includes a triangular cutout positioned between the clamping surfaces of the first and the second beams.

In certain aspects of the disclosure, the first beam is separated from the second beam when the ligation clip is in the open position.

In aspects of the disclosure, the first and second beams are mirror images of each other.

In some aspects of the disclosure, the first end portion of the first beam defines a first through bore, the second end portion of the first beam includes a first finger, the first and portion of the second beam includes a second finger, and the second end portion of the second beam defines a second through bore, and the first finger is received within the first through bore and the second finger is received within the second through bore to retain the ligation clip in the clamped position.

In certain aspects of the disclosure, the clamping surfaces of each of the first and second beams include spaced protrusions that are positioned to center the ligation clip on an anvil shaft of a circular stapling device.

In aspects of the disclosure, the clamping surfaces of each of the first and second beams define a circular recess that is positioned to center the ligation clip on an anvil shaft of a circular stapling device.

In some aspects of the disclosure, the ligation clip is formed of a polymeric material.

Other aspects of the disclosure are directed to a ligation clip including a first beam and a second beam. The first beam has first and second end portions and a first clamping surface and the second beam has first and second end portions and a second clamping surface. The first beam is movable in relation to the second beam to move the ligation clip from an open position to a clamped position. The first and second beams are formed from a polymeric material. In the clamped position, the first and second clamping surfaces of the first and second beams define an opening having a maximum diameter of from about 0.3 inches to about 0.6 inches to receive an anvil shaft of a circular stapling device and to secure a vessel about the anvil shaft.

Other aspects of the disclosure are directed to a method of performing an anastomosis including positioning an anvil assembly of a circular stapling device within a first vessel portion such that an anvil head of the anvil assembly is positioned within the first vessel portion and an anvil shaft of the anvil assembly extends from an end portion of the first vessel portion; using a first ligation clip to secure the end portion of the first vessel portion to the anvil shaft; positioning a shell assembly of the circular stapling device within a second vessel portion such that an anvil retainer of the circular stapling device extends from the second vessel portion towards the first vessel portion; using a second ligation clip to secure an end portion of the second vessel portion to the anvil retainer; securing the anvil shaft of the circular stapling device to the anvil retainer of the circular stapling device; and actuating the circular stapling device to secure the first vessel portion to the second vessel portion.

In aspects of the disclosure, actuating the circular stapling device moves the anvil head towards a shell assembly of the circular stapling device to compress the end portions of the first and second vessel portions between the anvil head of the anvil assembly and the shell assembly of the circular stapling device.

In some aspects of the disclosure, actuating the circular stapling device includes cutting tissue within the first and second vessel portions to separate the first and second ligation clips from the first and second vessel portions.

In certain aspects of the disclosure, using a first ligation clip to secure the end portion of the first vessel portion to the anvil shaft and using a second ligation clip to secure an end portion of the second vessel portion to the anvil retainer includes applying the first ligation clip about the anvil shaft with a clip applier and applying the second ligation clip about the anvil retainer with the clip applier.

Other features of the disclosure will be appreciated from the following description.

### BRIEF DESCRIPTION OF THE DRAWINGS

Various aspects of the disclosed are described herein below with reference to the drawings, wherein:

FIG. 1 is a side perspective view of a ligation clip for forming a purse string according to aspects of this disclosure with beams of the ligation clip separated from each other;

FIG. 2 is a side perspective view of the ligation clip shown in FIG. 1 in a clamped position;

FIG. 3 is a cross-sectional view taken through section line 3-3 of FIG. 2;

FIG. 4 is a side perspective view of another version of the ligation clip shown in FIG. 1 in the clamped position;

FIG. 5 is a side perspective view of yet another version of the ligation clip shown in FIG. 1 in the clamped position;

FIG. 6 is a side perspective view of yet another version of the ligation clip shown in FIG. 1 in the clamped position;

FIG. 7 is a side perspective view of yet another version of the ligation clip shown in FIG. 1 in the clamped position;

FIG. 8 is a side perspective view of yet another version of the ligation clip shown in FIG. 1 in an open position;

FIG. 9 is a side perspective view of the ligation clip shown in FIG. 8 in the clamped position;

FIG. 10 is a side perspective view of yet another version of the ligation clip shown in FIG. 1 in the open position;

FIG. 11 is a side perspective view of yet another version of the ligation clip shown in FIG. 1 in the open position;

FIG. 12 is a side cross-sectional view of another version of the ligation clip shown in FIG. 1 in the clamped position;

FIG. 13 is a side cross-sectional view of another version of the ligation clip shown in FIG. 1 in the clamped position;

FIG. 14 is a side cross-sectional view of another version of the ligation clip shown in FIG. 1 in the clamped position;

FIG. 15 is a flow chart for a method of performing an anastomosis procedure using the ligation clips illustrated above, a circular stapling device, and a ligation clip applier;

FIG. 16 is a side perspective view of an anvil assembly of a circular stapling device positioned within a lumen of a first vessel portion;

FIG. 17 is a side perspective view of the anvil assembly of a circular stapling device shown partly in phantom positioned within the lumen of the first vessel portion shown in FIG. 16 with an end of the first vessel portion moved inwardly towards an anvil shaft of the anvil assembly;

FIG. 18 is a side perspective view of the anvil assembly of a circular stapling device shown partly in phantom received within the first vessel portion shown in FIG. 17 and a clip applier with the ligation clip shown in FIG. 10 received between jaws of the clip applier and positioned about the end of the first vessel portion and the anvil shaft;

FIG. 19 is an enlarged view of the indicated area of detail shown in FIG. 16;

FIG. 20 is a side perspective view of the anvil member of a circular stapling device shown in FIG. 19 with the end portion of the first vessel portion clipped about the anvil shaft;

FIG. 21 is a side perspective view of a distal portion a circular stapling device with the anvil assembly shown in FIG. 20 positioned within the vessel portion shown in FIG. 20 with the end portion of the vessel portion clipped about the anvil shaft, and a shell assembly of the circular stapling device positioned within a second vessel portion with the end portion of the second vessel portion clipped about an anvil retainer of the circular stapling device with the anvil assembly coupled to the anvil retainer and the circular stapling device in an open position;

FIG. 22 is a side perspective view of a distal portion a circular stapling device with the anvil assembly shown in FIG. 20 positioned within the vessel portion shown in FIG. 20 with the end portion of the vessel portion clipped about the anvil shaft, and a shell assembly of the circular stapling device positioned within a second vessel portion with the end portion of the second vessel portion clipped about an anvil retainer of the circular stapling device with the anvil assembly coupled to the anvil retainer and the circular stapling device in a clamped position;

FIG. 23 is a side perspective view of the first and second vessel portions after the vessel portions have been joined; and

FIG. 24 is a cross-sectional view taken along section line 24-24 of FIG. 23.

### DETAILED DESCRIPTION

The disclosed ligation clips and method for applying the ligation clips to perform an anastomosis will now be described in detail with reference to the drawings in which like reference numerals designate identical or corresponding elements in each of the several views. However, it is to be understood that aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as distal, proximal, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel. As used herein, the term “purse string” is used generally to mean structure to secure end portions of vessel portions to be joined together to a circular stapling device.

The disclosed ligation clips are configured to be received about end portions of vessel portions to be joined together during an anastomosis procedure to secure the end portions to an anvil shaft and/or an anvil retainer of a circular stapling device. The use of the disclosed ligation clips to perform this function obviates the need to form a purse suture in the end portions of the vessel portions during the anastomosis procedure and reduces the amount of time required to perform an anastomosis.

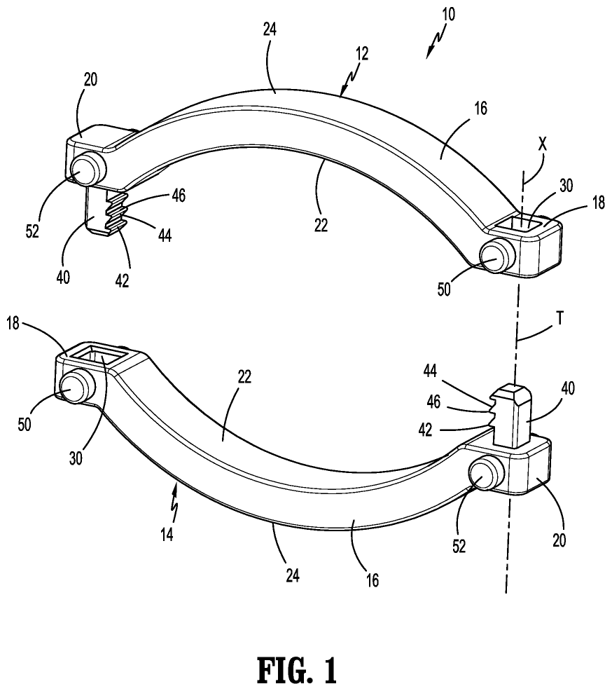

FIGS. 1 to 3 illustrate one version of a ligation clip for forming a purse string during an anastomosis procedure shown generally as ligation clip 10. The ligation clip 10 includes a first beam 12 and a second beam 14. In aspects of the disclosure, the first and second beams 12 and 14 are mirror images of each other. Each of the first and second beams 12 and 14 includes a body 16 that has a first end portion 18, a second end portion 20, an inner clamping surface 22, and an outer surface 24. The first end portion 18 of each of the first and second beams 12, 14 includes a through bore 30 defining a longitudinal axis “X” (FIG. 1) that extends from the inner clamping surface 22 of the body 16 to the outer surface 24 of the body 16. In one aspect of the disclosure, the through bore 30 is defined by a wall having a tooth 32 (FIG. 3). In certain aspects of the disclosure, the tooth 32 has a ramped surface 34 and a lock surface 36 that defines a plane that is substantially orthogonal to the longitudinal axis “X” of the through bore 30.

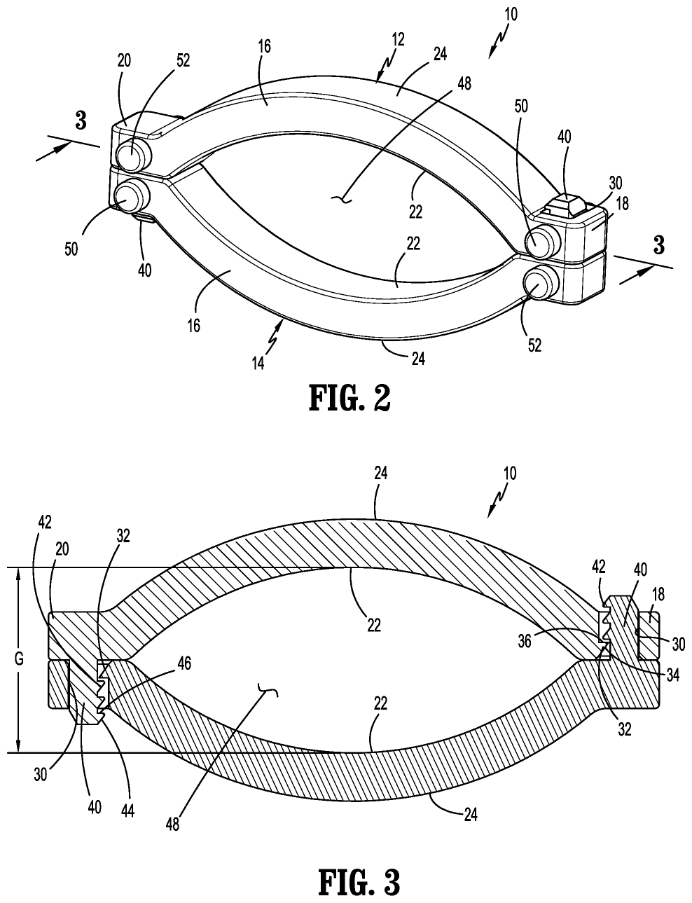

The second end 20 of the body 16 of each of the beams 12, 14 supports a finger 40 that defines an axis “T” (FIG. 1) that is substantially coaxial with the longitudinal axis “X” of the through bore 30 when the first and second beams 12 and 14 are secured together and the ligation clip 10 is in a clamped position (FIG. 2). The finger 40 and the through bore 30 form latching structure of the ligation clip 10 that functions to retain the ligation clip 10 in clamped positions. In certain aspects of the disclosure, each of the fingers 40 includes one or more teeth 42 that sequentially engage the tooth 32 located on the wall defining the through bore 30 of a respective beam 12, 14 to secure the first and second beams 12, 14 together in a plurality of distinct clamped positions. In each of the distinct clamped positions, a gap “G” (FIG. 3) is defined between the beams 12, 14. The size of the gap “G” varies depending on which of the teeth 42 is engaged with the tooth 32 to accommodate tissues of different thicknesses. In aspects of the disclosure, one tooth 32 is provided within the through bore 30 and a plurality of teeth 42 are provided on the finger 40. Each of the teeth 42 also has a ramped surface 44 (FIG. 3) and a lock surface 46 that defines a plane that is substantially orthogonal to the longitudinal axis “T” of the finger 40. Engagement between the locking surface 36 of the tooth 32 and the locking surface 46 of each one of the teeth 42 prevents inadvertent separation of the first and second beams 12 and 14 of the ligation clip 10 to retain the ligation clip 10 in the clamped position (FIG. 2). It is envisioned that a single tooth 42 can be provided on the fingers 40 and a plurality of teeth 32 can be provided within the through bore 30 to provide adjustability to the ligation clip 10.

The inner clamping surface 22 of the body 16 of each of the beams 12 and 14 is located between the through bore 30 and the finger 40 of the respective beams 12 and 14. The clamping surfaces 22 of the beams 12 and 14 extend along a common plane and are curved outwardly away from each other when the ligation clip 10 is in the clamped position (FIG. 2). As such, in the clamped position (FIG. 2), the ligation clip 10 defines an opening 48 (FIG. 2) that is positioned between the clamping surfaces 22 of the first and second beams 12 and 14. Although the opening 48 is shown to have an oval shape, other configurations are envisioned. The opening 48 defined by the ligation clip 10 when the ligation clip 10 is in the clamped position is dimensioned to securely clamp an end portion of a vessel portion to be joined about an anvil shaft and/or an anvil retainer of a circular stapling device as described below. In aspects of the disclosure, the maximum diameter of the opening 48 is from about 0.3 inches to about 0.6 inches. However, it is envisioned that the dimensions of the ligation clip 10 can be modified to accommodate different size circular stapling devices and tissue.

Although not shown, the clamping surfaces 22 of one or both beams 12 and 14 may include retention structure to minimize any likelihood of slippage of the ligation clip 10 in relation to tissue when the ligation clip 10 is applied to the tissue. The retention structure can be in the form of protrusions or recesses having a variety of different configurations.

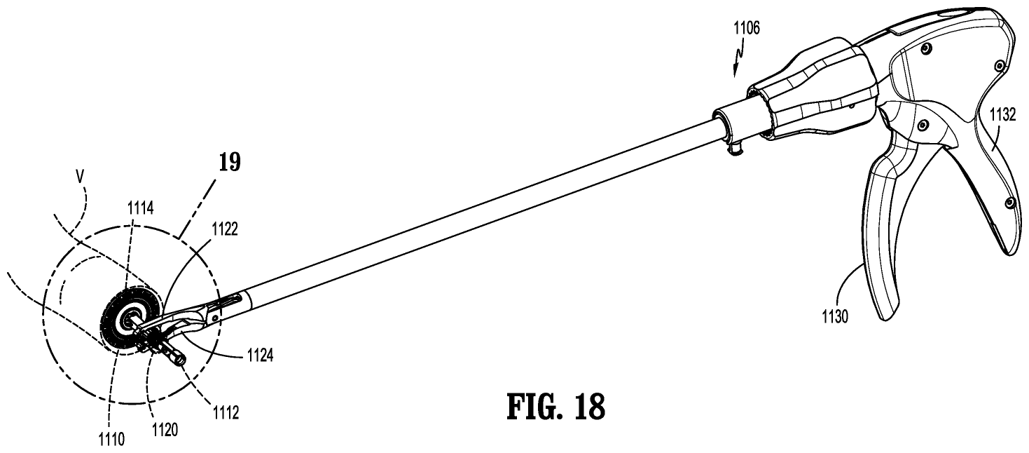

The first end portion 18 of the body 16 of the first and second beams 12 and 14 includes posts 50 and the second end portion 20 of the body 16 of the first and second beams 12 and 14 includes posts 52. The posts 50 and 52 have a substantially circular shape and are configured to be received in channels or recesses in a clip applier to secure the beams 12 and 14 to the clip applier (FIG. 18) and/or to retain the ligation clip 10 within jaws of the clip applier 1106 prior to application of the ligation clip 10 to tissue.

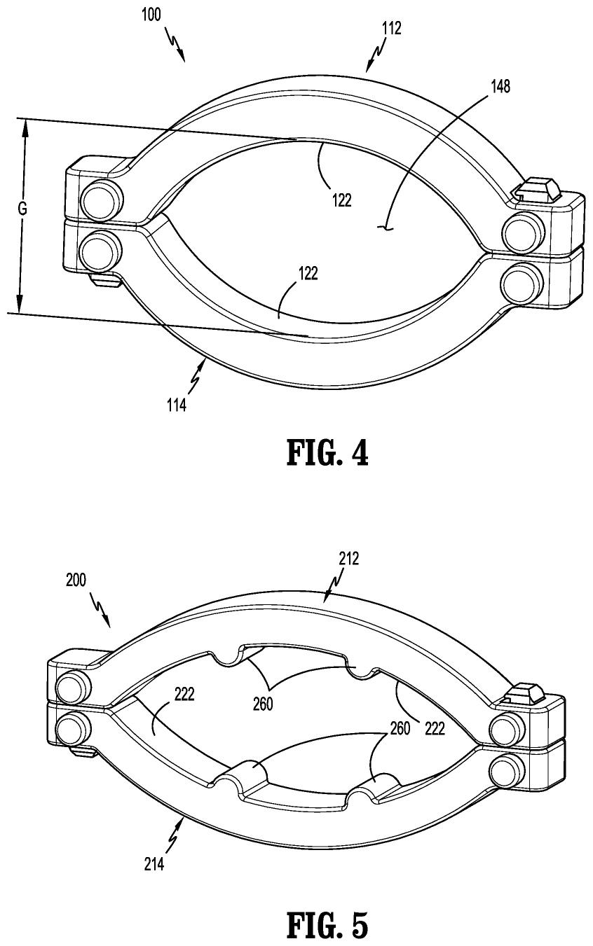

FIG. 4 illustrates another version of the disclosed ligation clip shown generally as ligation clip 100. The ligation clip 100 is substantially the same as ligation clip 10 except that the first and second beams 112 and 114 have a smaller radius of curvature such that the opening 148 defined between the clamping surfaces 122 of the beams 112 and 114 when the ligation clip 100 is in the clamped position is more circular than the opening 48 (FIG. 2) of the ligation clip 10.

FIG. 5 illustrates another version of the disclosed ligation clip shown generally as ligation clip 200. The ligation clip 200 is substantially the same as the ligation clip 10 (FIG. 2) described above except that the clamping surfaces 222 of the first and second beams 212 and 214 each include spaced protrusions 260. The spaced protrusions 260 are positioned on the clamping surfaces 222 to center the ligation clip 200 on an anvil shaft and/or anvil retainer of a circular stapling device. Although shown as semi-circular in shape, it is envisioned that the protrusions 260 can have other configurations including triangular, square, rectangular, etc.

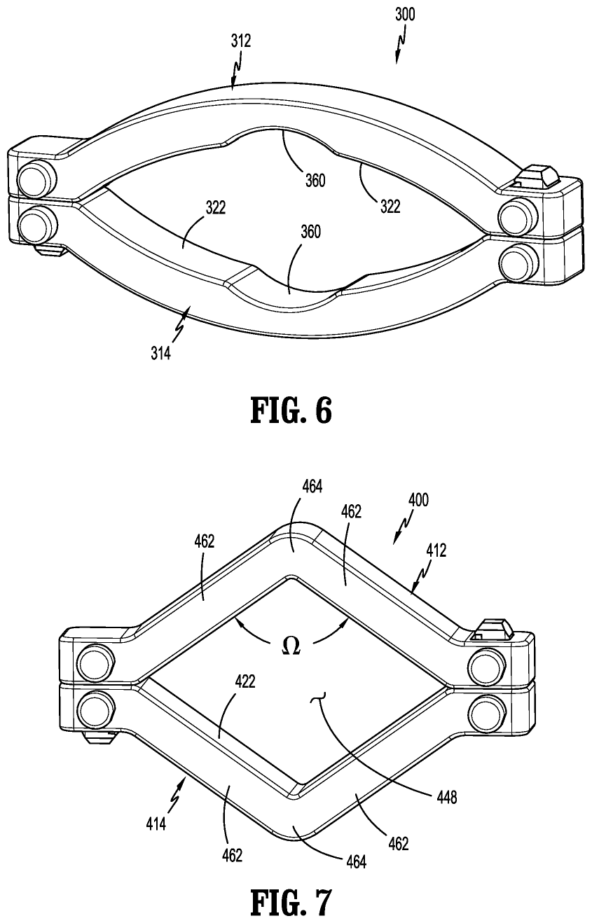

FIG. 6 illustrates another version of the disclosed ligation clip shown generally as ligation clip 300. The ligation clip 300 is substantially the same as the ligation clip 10 (FIG. 2) described above except that the clamping surfaces 322 of the first and second beams 312 and 314 each define a circular recess or concavity 360. The recesses 360 are centrally positioned on the clamping surfaces 322 of each of the first and second beams 312 and 314 to center the ligation clip 300 on an anvil shaft or anvil retainer of a circular stapling device. Although shown as semi-circular in shape, it is envisioned that the recesses 360 can have other configurations including triangular, square, rectangular, etc. It is also envisioned that each of the clamping surfaces 322 of the first and second beams 312 and 314 may include a plurality of recesses 360.

FIG. 7 illustrates another version of the ligation clip shown generally as ligation clip 400. The ligation clip 400 is substantially the same as ligation clip 10 except that the first and second beams 412 and 414 have a triangular shape such that the opening 448 defined between the clamping surfaces 422 of the first and second beams 412 and 414 is substantially rectangular or square shaped when the ligation clip 400 is in the clamped position. Each of the first and second beams 412 and 414 is formed from two linear sections 462 that are joined together at a corner 464 that defines an angle Ω. In aspects of the disclosure, angle Ω is from about 75 degrees to about 90 degrees.

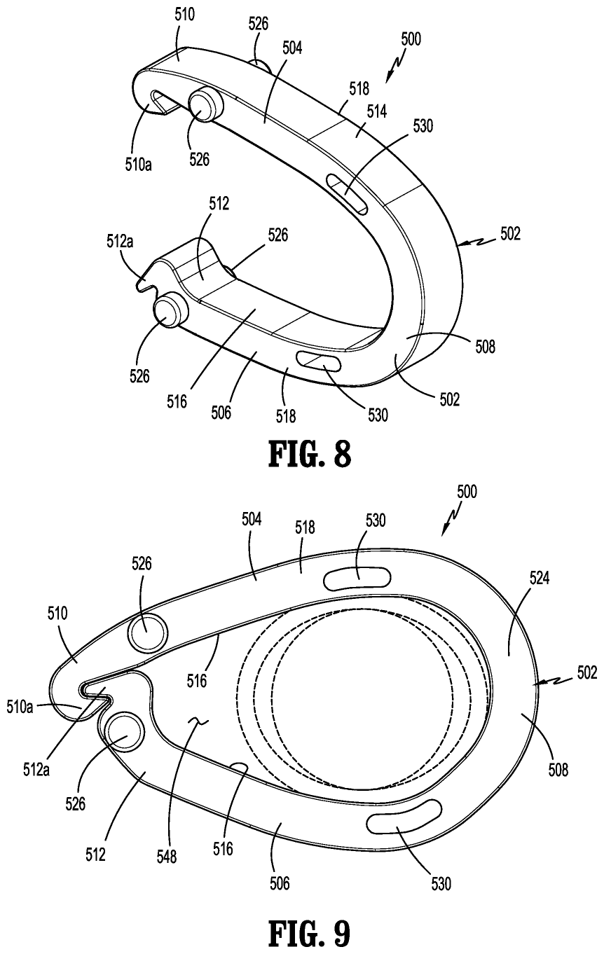

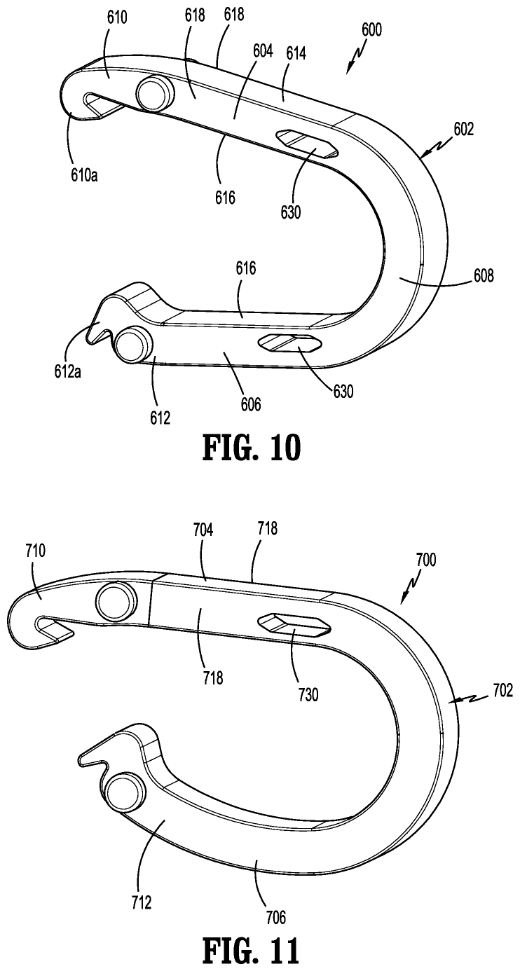

FIGS. 8 and 9 illustrate yet another version of the disclosed ligation clip which is shown generally as ligation clip 500. The ligation clip 500 includes a body 502 including a single unitary piece which has a C-shaped configuration and includes a first beam 504 and a second beam 506 that are joined by a central portion 508. The first beam 504 has a first end 510 and the second beam has a second end 512. The ligation clip 500 includes an outer surface 514, inner clamping surfaces 516, and side walls 518. Each of the ends 510 and 512 of the first and second beams 504 and 506 of the body 502 of the ligation clip 500 includes a hook shaped latch portion 510a and 512a, respectively. The latch portions 510a and 512a are movable into engagement with each other to retain the ligation clip 500 in a clamped position (FIG. 9). In the clamped position, the body 502 of the ligation clip 500 defines an opening 548. In some aspects of the disclosure, the opening 548 has a width that increases from the first and second ends 510 and 512 of the ligation clip 500 towards the central portion 508 of the ligation clip 500. The width of the opening 548 has a maximum diameter of from about 0.3 inches to about 0.6 inches.

In some aspects of the disclosure, the first and second ends 510 and 512 of the first and second beams 504 and 506 of the body 502 of the ligation clip 500 include posts 526. The posts 526 have a substantially circular shape and are configured to be received within recesses in jaws of a clip applier (FIG. 18) as described below to retain the ligation clip 500 within the clip applier (FIG. 18) prior to application of the ligation clip 500 to tissue. In other aspects of the disclosure, the body 502 of the ligation clip 500 defines channels 530 that extend between the side walls 518 of each of the first and second beams 504 and 506 of the ligation clip 500. The channels 530 increase the flexibility of the first and second beams 504 and 506 of the body 502 to allow the beams 504 and 506 of the body 502 to flex more easily between the open (FIG. 8) and clamped (FIG. 9) positions. In some aspects of the disclosure, the channels 530 are elongated and have semi-circular end portions.

FIG. 10 illustrates another version of the disclosed ligation clip shown generally as ligation clip 600. The ligation clip 600 is substantially like the ligation clip 500 (FIG. 9) and includes body 602 formed of a single unitary piece which has a C-shaped configuration and includes a first beam 604 having a first end 610, a second beam 606 having a second end 612, and a central portion 608 that interconnects the first and second beams 604 and 606. Each of the beams 604 and 606 of the body 602 includes an outer surface 614, inner clamping surfaces 616, and side walls 618. Each of the ends 610 and 612 of the first and second beams 604 and 606 of the body 602 includes a hook shaped latch portion 610a and 612a, respectively. The latch portions 610a and 612a are movable into engagement with each other to retain the ligation clip 600 in a clamped position. In the clamped position, the body 602 of the ligation clip 600 defines an opening (not shown). The ligation clip 600 differs from ligation clip 500 in that the first end 610 of the first beam 604 of the ligation clip 600 overhangs the second end 612 of the second beam 606 of the ligation clip 600 when the ligation clip 600 is in the open position to a greater extent than the ligation clip 500 (FIG. 9) and in that the channels 630 defined between the side walls 618 have a slightly different shape than the channels 530 (FIG. 9) of the ligation clip 500. The increased overhang of the first end 610 of the ligation clip 600 results in an opening that is more elongated than the opening 548 in the in the ligation clip 500 when the ligation clip 600 is in the clamped position. The channels 630 are also elongated but are defined by triangular end portions rather than semi-circular end portions as with the ligation clip 500 (FIG. 8)

FIG. 11 illustrates another version of the disclosed ligation clip shown generally as ligation clip 700. The ligation clip 700 is substantially the same as the ligation clip 600 except that the body 702 of the ligation clip 700 includes a single channel 730 that extends between the side walls 718 of the first and second beams 704 and 706 of the body 702 of the ligation clip 700. In aspects of the disclosure, the channel 730 is positioned in the first beam 704 of the ligation clip 700. The other components of the ligation clip 700 are as described regarding the ligation clip 600.

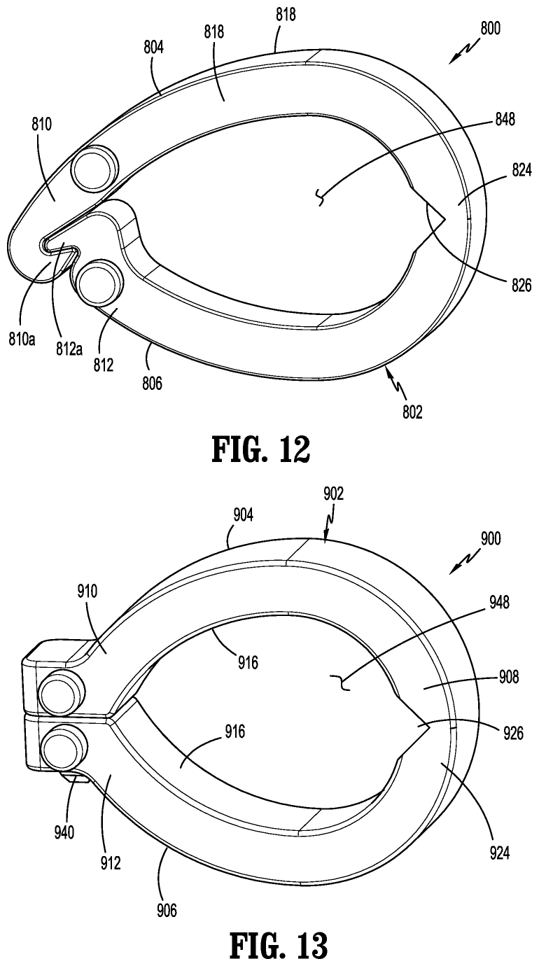

FIG. 12 illustrates another version of the disclosed ligation clip shown generally as ligation clip 800. The ligation clip 800 is substantially like the ligation clips 500, 600, and 700 described above except that the body 802 of the ligation clip 800 does not include a channel or channels that extend between the side walls 818 of the body 802. In contrast, the body 802 of the ligation clip 800 includes a central hinge portion 824 that is positioned on the central portion 808 of the body 802 of the ligation clip 800 between the clamping surfaces 816 of the first and second beams 804 and 806. The central hinge portion 824 includes a triangular cutout 826 that allows the first and second beams 804 and 806 to more easily pivot towards each other from the open position to the clamped position to define the opening 848. The first and second ends 810 and 812 of the first and second beams 804 and 806 of the body 802 include hook members 810a and 812a that engage each other to secure the ligation clip 800 in the clamped position.

FIG. 13 illustrates yet another version of the disclosed ligation clip shown generally as ligation clip 900. The ligation clip 900 is substantially similar to the ligation clip 800 (FIG. 12) except that the hook members 810a and 812a on the first and second ends 810 and 812 of the first and second beams 804 and 806 are replaced with a through bore, e.g., through bore 30 (FIG. 1) and a finger, e.g., finger 40 (FIG. 1). As described above regarding ligation clip 10 (FIG. 1), the finger 940 is received within the through bore (not shown) to secure the ligation clip 900 in a plurality of distinct clamped positions. The body 902 also includes a central hinge portion 924 that is positioned on the central portion 908 of the body 902 of the ligation clip 900. The central hinge portion 924 includes a triangular cutout 926 that is positioned between the clamping surfaces 916 of the first and second beams 904 and 906 of the ligation clip 900 that allows the first and second beams 904 and 906 to more easily pivot towards each other from the open position to the clamped position to define the opening 948. The body 902 of the ligation clip 900 defines a more circular opening 948 than that illustrated above regarding ligation clip 800 (FIG. 12).

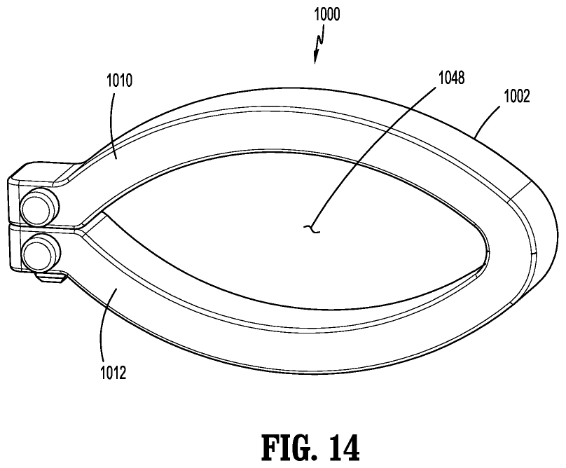

FIG. 14 illustrates another version of the disclosed ligation clip shown generally as ligation clip 1000. The ligation clip 1000 is substantially like the ligation clip 900 except that the radius of curvature of the first and second beams 1004 and 1006 of the body 1002 of the ligation clip 1000 is increased to define a more oval shaped opening 1048. The body 1002 of the ligation clip 1000 also does not include a triangular cutout to increase flexibility.

Each of the ligation clips described above is formed of a resilient material and is movable from an open position in which the first and second ends of the ligation clip define a mouth or opening to a clamped position in which the first end portion is releasably secured to the second end portion to define an opening within the ligation clip. In aspects of the disclosure, the opening is dimensioned to receive an anvil shaft or anvil retainer (hereinafter “shaft”) of a circular stapling device to secure end portions of vessel portions to be joined to the shaft during an anastomosis procedure. In some aspects of the disclosure, the ligation clips are formed of a polymeric material such as polyethylene terephthalate (PET), polybutylene terephthalate (PBT), polyoxymethylene, or other thermoplastic materials having similar properties that can be injection molded, extruded, or otherwise processed. It is also envisioned that the ligation clips described herein could be formed from other materials including metals.

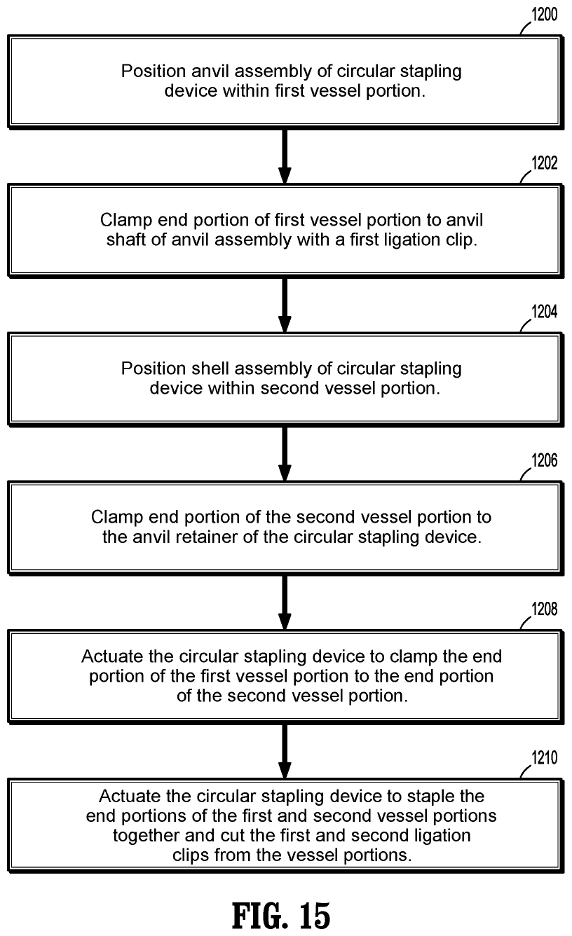

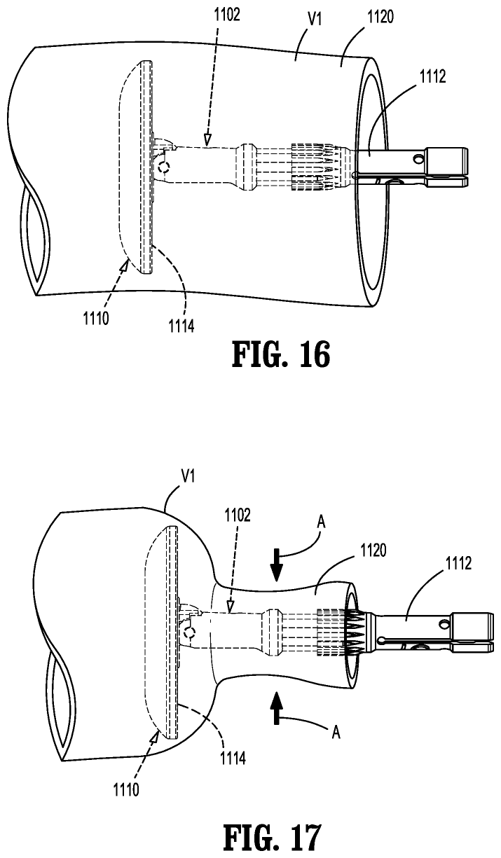

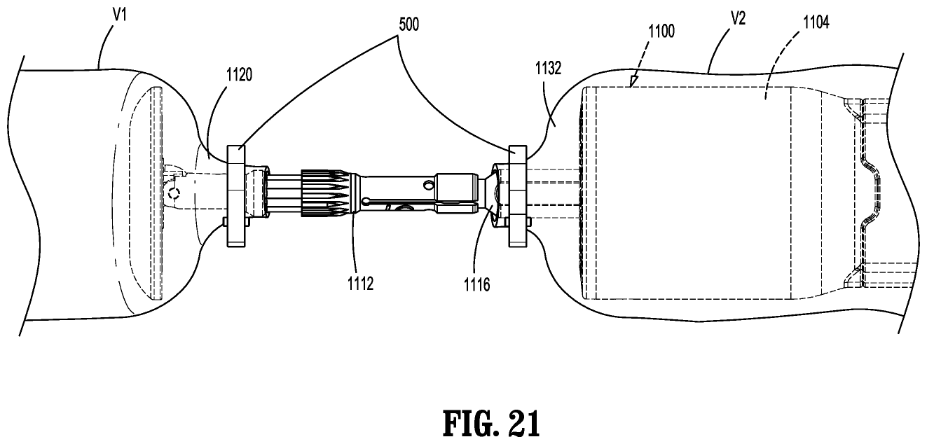

FIGS. 15-24 illustrate a method of performing an anastomosis procedure using the ligation clip 500 (FIG. 8), a circular stapling device 1100 (FIG. 21) including an anvil assembly 1102 (FIG. 16) and a shell assembly 1104 (FIG. 21), and a clip applier 1106 (FIG. 18). Anastomosis is an artificial connection of two vessel portions made by a surgeon and is required when a diseased portion of a vessel, e.g., artery, vein, or intestine, is removed from the vessel and the remaining vessel portions are rejoined. For example, during a colectomy procedure, a diseased portion of the colon is removed and end portions of the colon on opposite sides of the diseased portion of the colon are rejoined using a circular stapling device 1100. Such a procedure using the disclosed ligation clips is described below.

FIGS. 16 and 17 illustrate placement of an anvil assembly 1102 of a circular stapling device 1100 (FIG. 21) into a first vessel portion “V1” (Step 1200, FIG. 15) after a diseased portion (not shown) of a vessel is removed from an existing vessel (not shown), e.g., the colon, leaving first and second end portions of the vessel “V1” and “V2” (FIG. 21) that must be rejoined. In order to perform an anastomosis of the vessel portions “V1” and V2”, the anvil assembly 1102 of the circular stapling device 1100 is positioned within the first vessel portion “V1”. The anvil assembly 1102 includes an anvil head 1110 and an anvil shaft 1112. The anvil head 1110 defines a staple forming surface 1114 and the anvil shaft 1112 is configured to releasably engage an anvil retainer 1116 (FIG. 21) of the circular stapling device 1110. The anvil assembly 1102 is positioned within the first vessel portion “V1” such that the staple forming surface 1114 of the anvil head 1110 faces the second vessel portion “V2” and the anvil shaft 1112 projects from the end portion 1120 of the first vessel portion “V1”. When the anvil assembly 1102 is properly positioned within the first vessel portion “V1”, the end portion 1120 of the first vessel portion “V1” is moved inwardly in the direction of arrows “A” in FIG. 17 about the anvil shaft 1112. This can be accomplished using the clinician's fingers, using a grasper, or the like.

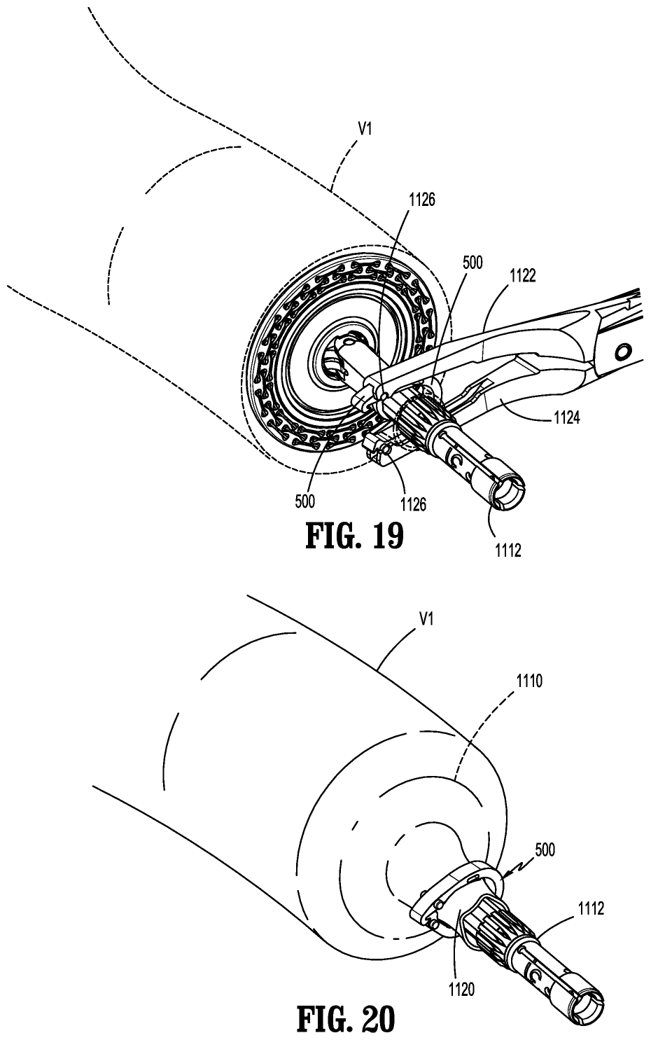

FIGS. 18-20 illustrate application of the ligation clip 500 about the end portion 1120 of the first vessel portion “V1” (Step 1202, FIG. 15) to secure the end portion 1120 to the anvil shaft 1112. When the end portion 1120 of the first vessel portion “V1” is positioned about the anvil shaft 1112, the clip applier 1106 is used to apply a ligation clip, e.g., clip 500 (FIG. 8), about the end portion 1120 of the first vessel portion “V1” and about the anvil shaft 1112 to secure the end portion 1120 of the first vessel portion “V1” to the anvil shaft 1112. The clip applier 1106 includes first and second jaws 1122 and 1124 (FIG. 19) that support the ligation clip 500. Each of the jaws 1122 and 1124 includes a recess 1126 that receives the posts 526 of the ligation clip 500 to support and retain the ligation clip 500 within the jaws 1122 and 1124. Once the jaws 1122 and 1124 of the clip applier 1126 are positioned about the anvil shaft 1112 and the end portion 1120 of the vessel portion “V1”, the clip applier 1106 can be actuated by moving a trigger 1130 of the clip applier 1106 towards a stationary handle 1132 of the clip applier 1106 to clamp the ligation clip 500 about the anvil shaft 1112 and secure the end portion 1120 of the first vessel portion “V1” to the anvil shaft 1112 (FIG. 20).

FIG. 21 illustrates the circular stapling device 1100 with the anvil assembly 1102 secured to the anvil retainer 1116 of the circular stapling device 1100. During the anastomosis procedure, the shell assembly 1104 is positioned within the second vessel portion “V2” (Step 1204, FIG. 15) such that the anvil retainer 1116 extends from the end portion 1132 of the second vessel portion “V2”. The end portion 1132 of the second vessel portion “V2” is moved inwardly about the anvil shaft 1112. As stated above, this can be accomplished using the clinician's fingers, using a grasper, or the like. Once the end portion 1132 of the second vessel portion “V2” is positioned about the anvil retainer 1116, a clip 500 can be applied about the end portion 1132 of the second vessel portion “V2” (Step 1206, FIG. 15) and the anvil retainer 1116 to secure the end portion 1132 of the second vessel portion “V2” to the anvil retainer 1116 (FIG. 21). It is noted that the end portions 1120 and 1132 of the first and second vessel portions “V1” and “V2” can be secured about the anvil shaft 1112 and the anvil retainer 1116, respectively while the anvil assembly 1102 is attached to the anvil retainer 1116 or separated from the anvil retainer 1116.

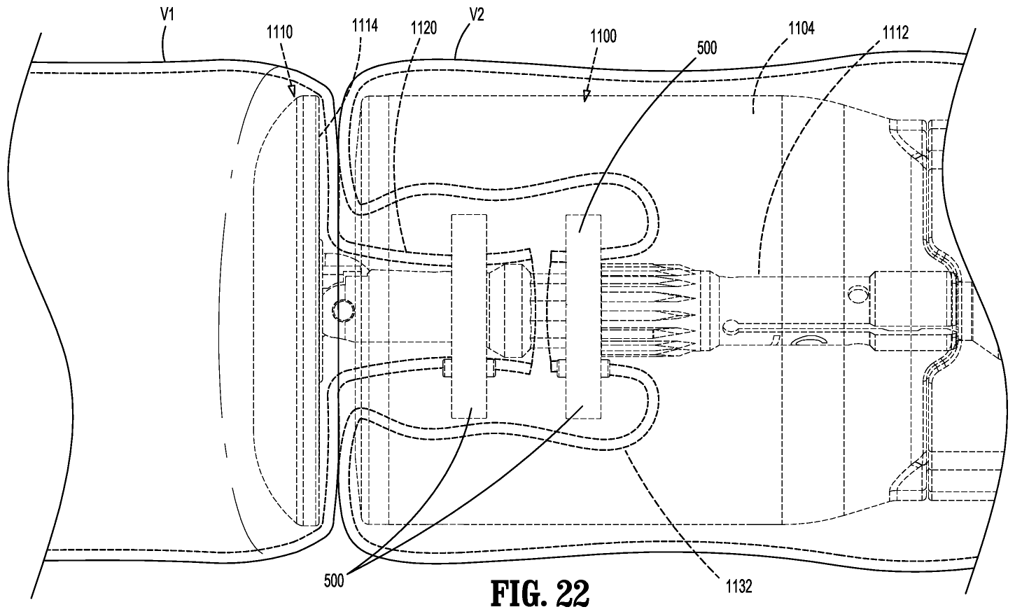

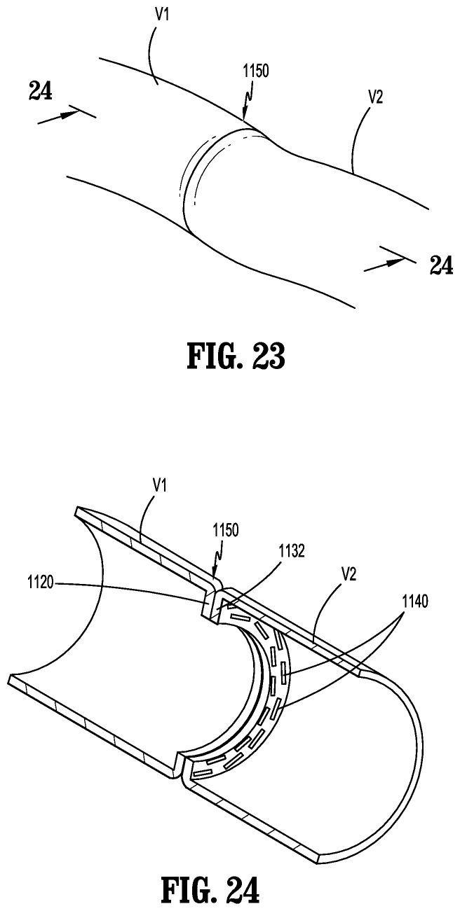

FIG. 22 illustrates the circular stapling device 1100 after the circular stapling device 1100 is moved to a clamped position in which the end portions 1120 and 1132 of the first and second vessel portions “V1” and “V2” are clamped between the staple forming surface 1114 of the anvil head 1110 and the shell assembly 1104 of the circular stapling device 1100 (Step 1208, FIG. 15). Once the end portions 1120 and 1132 of the first and second vessel portions “V1” and “V2” are clamped together between the staple forming surface 1114 of the anvil head 1110 and the shell assembly 1104, the circular stapling device 1100 can be actuated (Step 1210, FIG. 15) to staple the end portions 1120 and 1132 together with staples 1140 (FIG. 24) to join the vessel portions “V1” and “V2” as shown in FIGS. 23 and 24 and to remove excess tissue and the ligation clips from within the joined vessel 1150 (FIG. 24). U.S. Pat. No. 7,303,106 describes operation of a circular stapling device suitable for use in this anastomosis procedure.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A ligation clip comprising:
a first beam having first and second end portions and a first clamping surface; and
a second beam having first and second end portions and a second clamping surface, the first beam being movable in relation to the second beam to move the ligation clip from an open position to a clamped position, wherein in the clamped position, the first and second clamping surfaces of the first and second beams define an opening that is dimensioned to receive an anvil shaft of a surgical stapling device to secure a vessel about the anvil shaft.

2. The ligation clip of claim 1, wherein each of the first end portions of the first and second beams include a latch portion, the latch portions of the first and second beams engaging each other when the ligation clip is in the clamped position to retain the ligation clip in the clamped position.

3. The ligation clip of claim 2, wherein the diameter of the opening defined by the clamping surfaces of the first and second beams of the ligation clip when the ligation clip is in the clamped position is from about 0.3 inches to about 0.6 inches.

4. The ligation clip of claim 3, wherein the opening has a circular configuration.

5. The ligation clip of claim 3, wherein the opening has an oval configuration.

6. The ligation clip of claim 1, wherein the opening has a rectangular configuration.

7. The ligation clip of claim 2, wherein each of the latch portions is hook-shaped.

8. The ligation clip of claim 1, wherein the first and second beams are coupled together by a central hinge portion.

9. The ligation clip of claim 8, wherein the central hinge portion includes a triangular cutout positioned between the clamping surfaces of the first and the second beams.

10. The ligation clip of claim 1, wherein the first beam is a separated from the second beam when the ligation clip is in the open position.

11. The ligation clip of claim 10, wherein the first and second beams are mirror images of each other.

12. The ligation clip of claim 11, wherein the first end portion of the first beam defines a first through bore, the second end portion of the first beam includes a first finger, the first and portion of the second beam includes a second finger, and the second end portion of the second beam defines a second through bore, the first finger being received within the first through bore and the second finger received within the second through bore to retain the ligation clip in the clamped position.

13. The ligation clip of claim 1, wherein the clamping surfaces of each of the first and second beams include spaced protrusions, the spaced protrusions positioned to center the ligation clip on an anvil shaft of a circular stapling device.

14. The ligation clip of claim 1, wherein the clamping surfaces of each of the first and second beams define a circular recess, the circular recesses positioned to center the ligation clip on an anvil shaft of a circular stapling device.

15. The ligation clip of claim 1, wherein the ligation clip is formed of a polymeric material.

16. A ligation clip comprising:
a first beam formed of a polymeric material having first and second end portions and a first clamping surface; and
a second beam formed of a polymeric material having first and second end portions and a second clamping surface, the first beam movable in relation to the second beam to move the ligation clip from an open position to a clamped position, wherein in the clamped position, the first and second clamping surfaces of the first and second beams define an opening having a diameter of from about 0.3 inches to about 0.6 inches to receive an anvil shaft of a surgical stapling device and to secure a vessel about the anvil shaft.

17. A method of performing an anastomosis comprising:
positioning an anvil assembly of a circular stapling device within a first vessel portion such that an anvil head of the anvil assembly is positioned within the first vessel portion and an anvil shaft of the anvil assembly extends from an end portion of the first vessel portion;
using a first ligation clip to secure the end portion of the first vessel portion to the anvil shaft;
positioning a shell assembly of the circular stapling device within a second vessel portion such that an anvil retainer of the circular stapling device extends from the second vessel portion towards the first vessel portion;
using a second ligation clip to secure an end portion of the second vessel portion to the anvil retainer;
securing the anvil shaft of the circular stapling device to the anvil retainer of the circular stapling device; and
actuating the circular stapling device to secure the first vessel portion to the second vessel portion.

18. The method of claim 17, wherein actuating the circular stapling device moves the anvil head towards a shell assembly of the circular stapling device to compress the end portions of the first and second vessel portions between the anvil head of the anvil assembly and the shell assembly of the circular stapling device.

19. The method of claim 18, wherein actuating the circular stapling device includes cutting tissue within the first and second vessel portions to separate the first and second ligation clips from the first and second vessel portions.

20. The method of claim 19, wherein using a first ligation clip to secure the end portion of the first vessel portion to the anvil shaft and using a second ligation clip to secure an end portion of the second vessel portion to the anvil retainer includes applying the first ligation clip about the anvil shaft with a clip applier and applying the second ligation clip about the anvil retainer with the clip applier.
